(19) United States
(12) Patent Application Publication
(10) Pub. No.: US 2026/0272782 A1
(43) Pub. Date: Sep. 17, 2026

(54) Direct-From-Vial Medication Transfer Device

(71) Applicant: BIO HEALTH FRONTIERS, INC., El Dorado Hills, CA (US)

(72) Inventors: Paul Lambert, El Dorado Hills, CA (US); Carlos Gutierrez, El Dorado Hills, CA (US); Travis Schraeder, El Dorado Hills, CA (US); Neal de Beer, El Dorado Hills, CA (US); JonPaul Lambert, El Dorado Hills, CA (US); Kenneth Tallo, El Dorado Hills, CA (US); Samuel Miller, El Dorado Hills, CA (US)

(21) Appl. No.: 19/248,181

(22) Filed: Jun. 24, 2025

Related U.S. Application Data
(63) Continuation-in-part of application No. 19/082,007, filed on Mar. 17, 2025.

(51) Int. Cl. A61J 1/20; A61M 5/142; A61M 39/24

(52) U.S. Cl. CPC A61J 1/2096; A61J 1/2013; A61J 1/2082; A61M 5/14212; A61M 39/24; A61M 2205/273

(57) ABSTRACT

A medication transfer device for use with a medication container such as a vial includes a first interface, a second interface, and one or more piercing elements. The first interface can be on a first end have a shape and size to couple to an infusion pump. The second interface can be on a second end and have a shape and size to secure the vial. The one or more piercing elements can define both at least a portion of (i) an inlet channel in fluid communication with a pressure source generated by the infusion pump when the first interface is coupled to the infusion pump and (ii) an outlet channel in direct fluid communication with an infusion set delivering medication from the vial to a patient.

### RELATED APPLICATION

This application is a continuation-in-part of U.S. patent application Ser. No. 19/082,007 filed on Mar. 17, 2025, the contents of which are hereby incorporated by reference.

### TECHNICAL FIELD

The subject matter described herein pertains to devices and implements designed for the precise infusion of medications, such as immunoglobulin, directly from medication containers, including vials.

### BACKGROUND

Vials serve as the primary containers for the storage and administration of liquid medications in clinical and pharmaceutical settings. The process of administering medication from a vial typically involves several precise steps, such as injecting air into the vial to equalize internal pressure and facilitate the withdrawal of the liquid. This procedure demands specialized training and proficiency to ensure proper technique and to minimize the risk of complications. A substantial proportion of vials are intended for single-use applications; improper handling or deviation from established protocols can compromise the sterility of the vial, thereby increasing the risk of infection. Although multi-dose vials are formulated with preservatives to inhibit microbial growth, they remain susceptible to contamination if strict aseptic techniques are not consistently observed. Contamination may occur through various mechanisms, including re-accessing the vial with a needle or syringe that has previously been used or has come into contact with non-sterile surfaces. These challenges highlight the critical importance of proper handling and underscore the ongoing need for improved systems or devices to enhance safety and reduce the likelihood of contamination during medication administration.

The conventional method for withdrawing medication from a vial employs a manual, syringe-based technique. Initially, the user retracts the syringe plunger to draw in a volume of air equivalent to or slightly exceeding the intended medication dose. The needle is then inserted through the vial's rubber stopper, and the air is injected into the vial to equalize internal pressure. Subsequently, the vial is inverted, ensuring that the needle tip remains immersed in the liquid medication. The desired dose is withdrawn by gradually pulling back the syringe plunger. To remove any air bubbles, the syringe may be gently tapped, allowing bubbles to rise to the top and be expelled back into the vial. Once the medication has been accurately drawn, the syringe is withdrawn from the vial and is ready for administration to the patient, either directly or through an intermediate delivery system.

### SUMMARY

In a first aspect, a medication infusion system includes a housing having a shape and size so as to define a medication container adapter interface. A medication transfer device is provided which has a shape and size to interface with the medication container adapter interface and with a medication container. The medication container has a medication chamber encapsulating fluid. The medication transfer device includes a first piercing element defining a portion of an inlet channel and a second piercing element defining a portion of an outlet channel. The first piercing element and the second piercing element are configured to pierce a surface of the medication container (e.g., a membrane such as a stopper, etc.) when it is secured in the medication container adapter. The outlet channel can, in some variations, be directly coupled to an infusion set for directly administrating medication to a patient. A pressure source is encapsulated within the housing which, when active, expels gas through the inlet channel into the medication container causing fluid within the medication chamber to be expelled out of the outlet channel for delivery to a patient. A control unit in communication with the pressure source selectively sends signals to the pressure source to control infusion parameters.

A length of the first piercing element can extend further into the medication container as compared to a length of the second piercing element. In other variations, a length of the first piercing element is equal to a length of the second piercing element.

The medication transfer device, in some variations, is integral to the housing. In other variations, the medication transfer device is selectively detachable from the housing and optionally disposable (i.e., intended for one-time use).

A pressure sensor within the housing can generate signals to indirectly measure or otherwise characterize pressure within the medication container. The control unit can be further in communication with the pressure sensor to selectively send the signals to the pressure source to control infusion parameters based on the measured pressure.

The medication container can take varying forms such as a vial.

A one way valve can be provided within or upstream from the inlet channel to prevent fluid within the medication container from exiting the medication container through the inlet channel.

A stop valve can be provided within or downstream from the outlet channel to selectively prevent fluid within the medication container from flowing downstream from the outlet channel. The stop valve can be an electromechanical valve coupled or in communication with the control unit. In some variations, the medication transfer device includes a first electrical conduit to the stop valve which is positioned so as to electrically connected to a second electrical conduit on the infusion pump which is connected to the control unit. The stop valve, some variations, is a duckbill valve.

A bubble eliminator can be provided within or downstream from the outlet channel which includes a bubble chamber and a hydrophobic air-permeable membrane through which bubbles within the bubble chamber pass. The bubble eliminator can form part of the medication transfer device. In other variations, the bubble eliminator is downstream from the medication transfer device. The bubble eliminator can be adjacent or form part of a needle set.

In some variations, a terminus of the outlet channel includes a tubing set interface configured to secure the infusion set. The tubing set interface can be, for example, a Luer lock and the infusion set can have a corresponding Luer lock. The infusion set can be one or more of a subcutaneous infusion set, an intravenous infusion set, or a needle cannula set for an implanted septum.

A filter can be disposed at some point intermediate the infusion pump and the medication container so as to purify or otherwise filter gas prior to introduction into the medication container. In some variations, the filter is adjacent to the inlet channel and can form part of an outer surface of the medication transfer device. In other variations, the filter is within the inlet channel (i.e., extends across a cross-section, etc.).

The pressure source can take varying forms. For example, the pressure can be a pneumatic pump. In some variations, the pneumatic pump is a piezoelectric pump. In some variations, the pressure source is a mechanical pump such as a manually-activated mechanical pump. The pressure source can include two or more pumps (e.g., one providing positive pressure and one providing negative pressure, etc.). The pressure source can be a bi-directional pump which, in response to a first signal from the control unit, exerts positive pressure and, in response to a second signal from the control unit, creates a vacuum.

The control unit can include a microprocessor and memory and can interface with the various components forming part of the infusion device in order to monitor, assess, and control infusion. The control unit can, for example, control infusion parameters using a locally stored look up table which correlates factors such as measured pressure, a geometry of the medication container, a viscosity of fluid (i.e., medication) contained within the medication container, and an infusion tubing set in communication with the orifice (i.e., the downstream infusion tubing and/or downstream needle set, etc.) with a predetermined flow rate.

The housing can also encapsulate a data store (e.g., memory, a memory device, etc.) to store infusion parameter measurements generated by the control unit. The infusion device and/or the control unit can include a central processing unit (CPU) which can be used to generate time measurements (e.g., time stamps, etc.) associated with the various pressure sensor measurements.

The infusion device can include a communications interface encapsulated within the housing and in communication with the data stored and the control unit. The communications interface can transmit data stored in the memory characterizing the infusion parameter measurements to a remote computing device. The communications interface can provide wired and/or wireless communications to external computing devices (including network-connected medical devices and equipment, mobile phones, tablets, patient monitors, hospital data systems, computers, etc.). In some variations, the communications interface is bidirectional and receives signals from one or more external network-connected medical devices associated with a patient (i.e., the person receiving the fluid expelled from the orifice of the medication container, etc.). The remote computing device can send signals, by way of the communications interface, to cause the control unit initiate and stop expulsion of the fluid from the medication container.

The transmitted data can include fluid flow rate information or values to calculate the fluid flow rate information. The fluid flow rate information can be one or more of: time of infusion, fluid flow rate, fluid pressure profile, mode of operation by the pressure source, and patient compliance statistics.

The control unit can estimate and correlate detected pressure with one or more of volume infused, flow rate, and therapy settings relative to a type of the fluid and/or an infusion protocol (e.g., a programmed infusion protocol, etc.).

The control unit can be configured to send signals to the pressure source to change a pressure limit from a first value to a second value.

The flow rate profile can be dynamically changed at all times. The control unit can be configured to send signals to the pressure source to cause the fluid to be expelled according to a controlled first flow rate profile as well as to change a fluid pressure profile at which the fluid is expelled from the first pressure profile to a second, different flow rate profile.

The control unit, based on the measured pressure, and fluid characterization data, can determine (i) a condition at which all of the fluid in the medication container has been expelled through the orifice based on a level of pressure detected in the pressure chamber, (ii) an occlusion condition within an infusion set coupled to the medication container based on a detected build up of pressure within the pressure chamber, and/or (iii) that the medication container has been removed from the medication container interface based on a detected release of pressure within the pressure chamber.

The infusion device can further include a proximity sensor in or adjacent to the medication container interface which is configured to detect presence or absence of a medication container within the medication container interface and transmit corresponding signals to the control unit.

The infusion device can further include a position sensor in or adjacent to the medication container interface which is configured to detect changes correlated to a volume of the medication chamber and transmit corresponding signals to the control unit.

The infusion device can include a pressure relief valve encapsulated within the housing and in communication with the control unit which, when activated in response to a signal from the control unit, causes the pressure chamber to vent resulting in a cessation of fluid out of the orifice. The pressure relieve valve can take varying forms including a solenoid valve.

The infusion device can include a mechanical pressure relief valve encapsulated within the housing which causes the expulsion of the fluid out of the orifice to cease when a predetermined physical parameter condition (e.g., pressure within the pressure chamber exceeds a threshold, etc.) is met.

The infusion device can include an expandable element (e.g., inflatable O-ring, etc.) disposed within (e.g., circumferentially around the mount, etc.) or adjacent to the medication container interface. The pressure source or a second, different pressure source within the housing can cause the expandable element to inflate to secure the medication container within the medication container interface.

In some variations, the infusion device includes an expandable element disposed within or adjacent to the medication container interface which, rather than being used to sure the medication container, can be inflated to exert a mechanical force on the first side of the medication container when placed within the medication container interface. This expandable element can be inflated by the pressure source or a second, different pressure source within the housing.

The infusion device can include an air port extending through an external surface of the housing having a fitting for communicating with an external tubing set or device for receiving a source of gas. The source of gas can be the pressure source or a second, different pressure source within the housing.

In some variations, the infusion device can also include a reference chamber, a first temperature sensor, and a second temperature sensor. The reference chamber has a known, fixed volume. The first temperature sensor can be within the housing and in communication with the control unit to measure temperature within the pressure chamber. The second temperature sensor can be within the housing and in communication with the control unit to measure temperature within the reference chamber. The infusion parameters, in such case, are further controlled based on values generated by the first temperature sensor and the second temperature sensor.

In an interrelated aspect, a medication infusion system includes a housing defining a medication container adapter interface. A medication transfer device has a shape and size to interface with the medication container adapter interface and with a medication container. The medication container includes a medication chamber encapsulating fluid. The medication transfer device includes at least one piercing element defining at least a portion of (i) an inlet channel and (ii) an outlet channel. The at least one piercing element is configured to pierce a surface of the medication container (e.g., membrane, stopper, etc.) when it is secured in the medication container adapter. The outlet channel can be directly coupled to an infusion set for administrating medication to a patient. A pressure source can be encapsulated within the housing which, when active, expels gas and/or fluid through the inlet channel into the medication container causing fluid within the medication chamber to be expelled out of the outlet channel for delivery to a patient. A control unit in communication with the pressure source can sends signals to the pressure source to control infusion parameters.

In some variations, there is a single piercing element that defines portions of both of the inlet channel and the outlet channel. In other variations, a first piercing element defines a portion of the inlet channel and a second, different piercing element defines a portion the outlet channel.

In a further interrelated aspect, a medication transfer device includes a first interface, a second interface, a first piercing element, and a second piercing element. The first interface is on a first end and has a shape and size to couple to an infusion pump. The second interface is on a second end and has a shape and size to secure a vial containing medication. The first piercing element defines at least a portion of an inlet channel in fluid communication with a pressure source generated by the infusion pump when the first interface is coupled to the infusion pump. The second piercing element defines at least a portion of an outlet channel in direct fluid communication with an infusion set delivering medication from the vial to a patient.

The medication transfer device can include a one way valve within the inlet channel to prevent fluid within the vial from exiting the vial through the inlet channel.

The medication transfer device can include a stop valve within the outlet channel to selectively prevent fluid within the vial from flowing downstream from the outlet channel into the infusion set. The stop valve can take varying forms including a duckbill valve or an electromechanical valve. With the latter, the medication transfer device can include a first electrical conduit connected to the electromechanical valve and positioned to interface with a second electrical conduit on the infusion pump, when the medication transfer device is coupled to the medication transfer device. The second electrical conduit can be in communication with a control unit within the infusion pump. The stop valve can also be a duckbill valve.

The medication transfer device can include a bubble eliminator within the outlet channel having a bubble chamber and a hydrophobic air-permeable membrane through which bubbles within the bubble chamber pass.

The medication transfer device can have a housing forming the first interface, the second interface, the first piercing element and the second piercing element and encapsulating the bubble eliminator.

A filter can be positioned at or adjacent to a first end of the inlet channel to purify gas passing therethrough. In other variations, the filter is positioned within the inlet channel.

In some variations, the first piercing elements comprises perforations or one or more grooves along at least a portion of its body positioned to facilitate expulsion of all liquid in the vial.

In an interrelated aspect, a medication transfer device includes a first interface, a second interface, and a single piercing element. The first interface can be on a first end have a shape and size to couple to an infusion pump. The second interface can be on a second end and have a shape and size to secure a vial containing medication. The single piercing element can define both at least a portion of (i) an inlet channel in fluid communication with a pressure source generated by the infusion pump when the first interface is coupled to the infusion pump and (ii) an outlet channel in direct fluid communication with an infusion set delivering medication from the vial to a patient.

In a further interrelated aspect, a medication transfer device includes a first interface, a second interface, and a single piercing element. The first interface in first end and has a shape and size to couple to a manually activated, mechanical pressure source. The second interface is on a second end (e.g., opposing side, etc.) having a shape and size to secure a vial containing medication. The first piercing element defines a portion of an inlet channel in fluid communication with a pressure source generated by the manually activated, mechanical pressure source when the first interface is coupled to the manually activated, mechanical pressure source. The second piercing element defines a portion of an outlet channel in direct fluid communication with an infusion set delivering medication from the vial to a patient.

The subject matter described herein provides many technical advantages. For example, the current subject matter provides a hand-held infusion pump that allows a user to safely self-administer medication from a vial according to a prescribed regimen without having to transfer from a vial to a different container reducing preparation steps, user error and contamination risks, thereby simplifying therapies.

The subject matter also provides functional advantages when compared to, constant pressure force pumps that will dynamically change their flow rate (up or down) based on a number of factors external to the pump and not the inherent design of the pump. The factors that affect the performance of mechanical pumps include the patient absorption rate design and geometry of the pump's accessories, and the variable drug viscosity—(a factor of temperature). On the other hand, pressure-based pumps have a similar dynamic effect on the flow rate. However, in pressure controlled pumps and with the current subject matter, there can be optimization of flow controlled by the pump through dynamic pressure adjustments as needed to optimize the infusion considering factors such as pump, accessories, patient and drug viscosity

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a diagram illustrating an infusion pump coupled to a medication container which, in turn, is connected to tubing and a needle set;

FIG. 2 is a diagram illustrating the infusion pump of FIG. 1 separated from the medication container, tubing, and needle set;

FIG. 3 is a diagram illustrating the infusion pump of FIG. 1;

FIG. 4 is a diagram illustrating an exploded view of the infusion pump of FIG. 1;

FIG. 5 is a first logical diagram illustrating components which can form part of the infusion pump of FIG. 1;

FIG. 6 is a second logical diagram illustrating components which can form part of the infusion pump of FIG. 1;

FIGS. 7-10 are diagrams illustrating a medication container adapter allowing differing medication container sizes to be secured to the infusion pump of FIG. 1;

FIGS. 11-14 are diagrams illustrating a medication transfer device allowing a vial to be secured to the infusion pump of FIG. 1;

FIG. 15 is a diagram illustrating a cross section of a first variation of the medication transfer device with the infusion pump and the vial;

FIG. 16 is a diagram illustrating a cross section of the first variation of the medication transfer device;

FIG. 17 is a diagram illustrating a cross section of a second variation of the medication transfer device with the infusion pump and the vial;

FIG. 18 is a diagram illustrating a cross-section of the second variation of the medication transfer device;

FIG. 19 is a diagram illustrating a cross section of a third variation of the medication transfer device with the infusion pump and the vial;

FIG. 20 is a diagram illustrating a cross section of the third variation of the medication transfer device;

FIG. 21 is a diagram illustrating a cross section of a fourth variation of the medication transfer device having a filter in an inlet channel to purify gas passing therethrough;

FIG. 22 is a diagram illustrating a cross section of the fourth variation of the medication transfer device having a filter positioned adjacent to the inlet channel to purify gas passing therethrough;

FIG. 23 is a diagram illustrating a cross section of a fourth variation of the medication transfer device with the infusion pump and the vial;

FIG. 24 is a diagram illustrating a cross section of the fourth variation of the medication transfer device;

FIG. 25 is a logic diagram illustrating aspects of the medication transfer device; and

FIG. 26 is a diagram illustrating aspects of a bubble eliminator.

Like reference symbols in the various drawings indicate like elements.

### DETAILED DESCRIPTION

The current subject matter relates to a compact, hand-held infusion pump designed to deliver fluids—such as medications—with high precision from a variety of medication containers. These containers may include syringes, flexible medication reservoirs with deformable portions, and rigid containers such as vials. This infusion pump is especially advantageous for applications like subcutaneous immunoglobulin (SCIg) therapy and similar treatments that require the controlled subcutaneous administration of medication at specific flow rates. Additionally, the current subject matter is directed to various components that enable direct administration of medication from a vial, eliminating the need for an intermediary container such as a syringe. In this context, “direct-from-vial” refers to systems in which medication is delivered straight from the vial to the patient without transfer to another container.

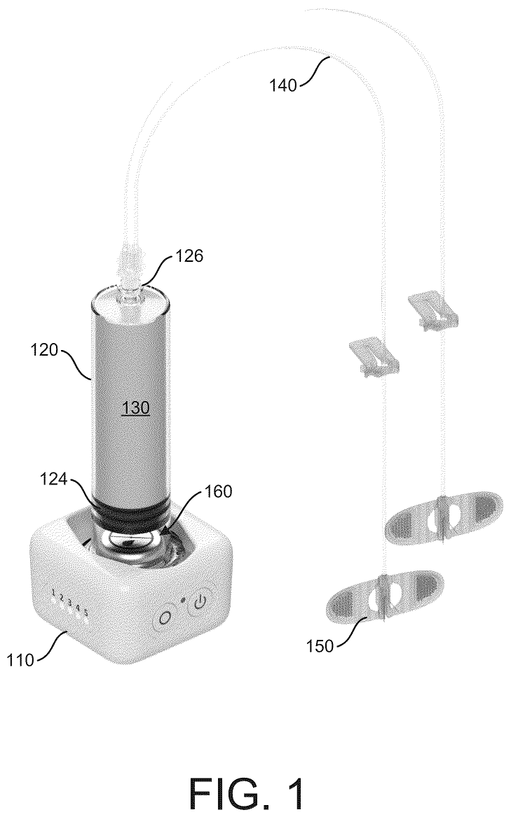

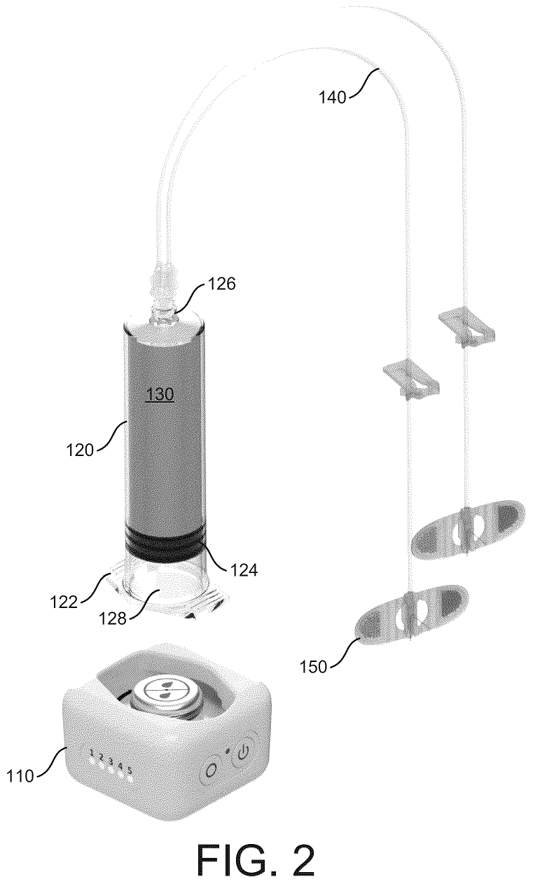

FIG. 1 is a diagram illustrating an arrangement in which an infusion pump 110 can receive and secure a medication container 120 and cause fluid (i.e., medication, etc.) contained therein to be infused to a patient by way of a tubing set 140 connected to or integrated with a needle set 150 (e.g., an intravenous needle set or a subcutaneous needle set, etc.). FIG. 2 is a diagram providing a view of the medication container 120 detached from the infusion pump 110. The medication container 120, in this variation, is a plunger-less syringe having one or more flanges 122, a rubber stopper 124, a medication chamber 130 (i.e., that portion of the medication container 120 in which fluid is housed), and an orifice 126. In some cases, the medication container 120 is a plunger-less syringe without flanges. Fluid contained within the medication chamber 130 is expelled when the rubber stopper 124 is translated in response to pressure (e.g., gas, etc.) generated by the infusion pump 110. An interior portion of the medication container 120 can be cylindrical/barrel shaped and include an opening 128 at an end opposite the orifice 126.

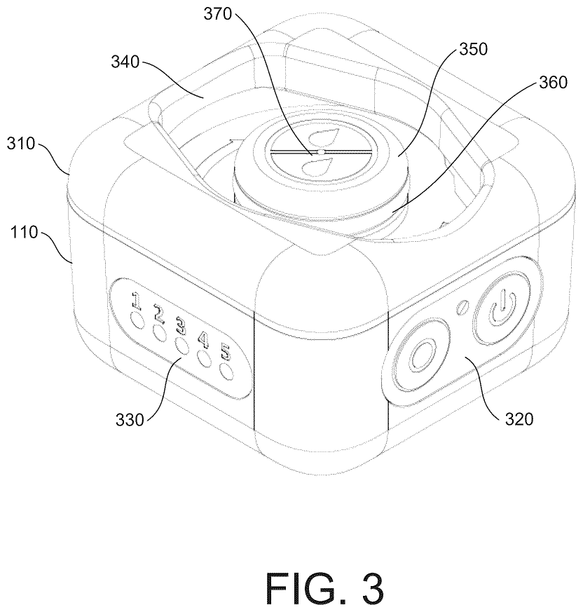

FIG. 3 is a diagram illustrating further details of the infusion pump 110. The infusion pump 110 can have a housing 310 with a shape and size to allow it to be handheld. In some variations, the housing 310 has a length and width of 8 cm or less and a height of 5 cm or less. The size of the housing can also vary based on the size of the utilized medication container (e.g., plunger-less syringe) and in some cases the housing 310 can be larger or in other cases it can be smaller. The external face of the housing 310 can include various manually-activated user control elements including power on/off buttons 320 and varying infusion setting elements 330. The infusion setting elements 330 can, for example, correspond to different infusion lengths (e.g., 1 minute, 5 minutes, 30 minutes, etc.) medications, prescription information, compatible supplies, and/or other infusion parameters (e.g., flow rate profiles, etc.). The infusion settings corresponding to the setting elements 330 can be programmable or otherwise modifiable by a user. For example, the infusion parameters for the settings elements 330 can be programmed by way of a remote computing device in communication with the infusion pump 110 (e.g., a mobile phone, tablet, remote computer, or other).

The housing 310 can form a medication container interface 340 which has a shape and size to receive and secure the medication container 120. The infusion pump 110 can additionally include a mount 350 (i.e., a medication container mount) having a shape and size to receive the opening 128 of the medication container 120. In the cases of the medication container 120 being a syringe or other receptacle with flanges 122, the medication container 120 can be positioned so that the mount 350 is inserted into the opening 128. The medication container 120 can then be rotated (e.g., clockwise) so that the flanges 122 contact or are otherwise secured by one or more securing elements forming part of the medication container interface 340. The securing elements can take varying forms including: locking tabs complementary to, for example, the flanges 122 and/or snaps to prevent the medication container 120 from rotating once it is secured. In some variations, the medication container interface 340 can include slots having a shape and size complementary to flanges 122 on the medication container 120 to allow for insertion and removal of the medication container 120 when not secured. In some variations, the medication container interface 310 can include a series of stacked circumferential grooves having depths corresponding to differently sized medication containers 120 (e.g., different syringe sizes, different flange sizes and positions, etc.).

The mount 350 can include one or more seals 360 (e.g., gaskets, O-rings etc.) on an outer circumference/surface to create an air-tight seal with the opening 128 of the medication container 120. The medication container 120 when secured to the medication container interface 340 forms a pressure chamber 160 in the open space within the opening 128 between the mount 350 and the plunger 124. In some variations, the one or more seals 360 are expandable so as to fit different sizes and/or tolerances of medication containers 120. For example, a pressure source 505 can cause the seal 360 to be inflated and thus expand to create the pressure chamber 160 within the medication container 120. In other variations, a second pressure source can be used to inflate the seal 360. The mount 350 can also include a valving mechanism 370 in which gas can be passed therethrough (as described in more detail below) into the pressure chamber.

In some variations, the mount 350 can have stacked and stepped ridges each having a decreasing diameter (relative to the distal portion of the mount 350) each having a corresponding seal 360. Such an arrangement can be used with differently sized medication containers 120 optionally without the need for any sort of adapter (such as those described below).

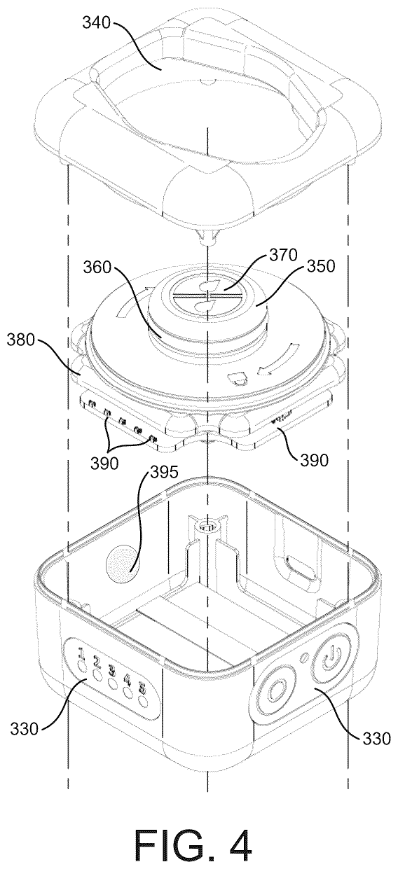

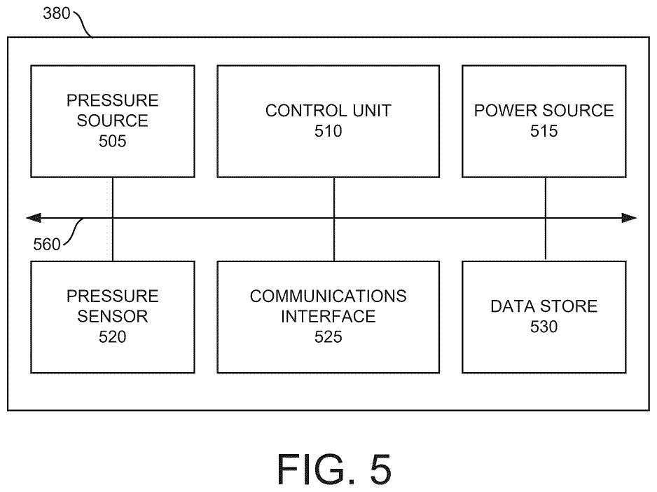

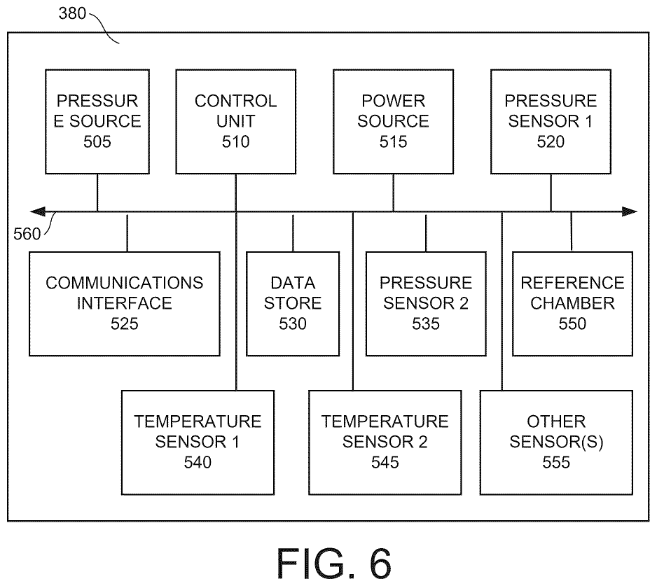

FIG. 4 is an exploded view of the infusion pump 110 which illustrates a component platform 380 on which various components 505-555 (illustrated logically in FIGS. 5 and 6) can be mounted and electrically connected, etc. In some cases, the various components 505-555 can be connected by way of a bus 560. Other configurations with direct communications amongst the components 505-555 can also be implemented. The component platform 380 allows for all of the components 505-555 to be encapsulated within the housing 310. Different mounting and connectivity arrangements can also be utilized provided that the components 505-555 are encapsulated within the housing 310.

The component platform 380 provides connectivity by way of the bus 560 amongst some or all of the components and secures a variety of components including, for example, a pressure source 505, a control unit 510, a power source 515, a pressure sensor 520, a communications interface 525 and/or a data store 530. In the variation of FIG. 6, the component platform 380 can include first and second pressure sensors 520, 535 and first and second temperature sensors 540, 545. A reference chamber 550 can also be placed on the component platform 370 which has a known volume and in which the second pressure sensor 535 and the second temperature sensor 545 are disposed therein (or otherwise positioned as to make measurements within the reference chamber 550). The component platform 370 can also have mechanical interfaces 390 which connect to or correspond with the on/off buttons 320 and the various external settings elements 330.

The pressure source 505 is a device or collection of device for generating pressure. The pressure source 505 can, for example, be a piezoelectric pneumatic gas pump or other pneumatic pump which is positioned or includes channels (i.e., tubes) to cause air (or other gas) be expelled into the pressure chamber 160 by way of the valving mechanism 370. In some variations, the pressure source 505 is a mechanical pump. An example mechanical pump which can be manually activated by a user is disclosed in U.S. patent application Ser. No. 17/774,100 published as US20220387708A1 and entitled: “Differential Pressure Fluid Delivery System”; the contents of which are hereby fully incorporated by reference. The pressure source 505 can operate within varying ranges such as 0.1 to 40 PSI, or between 0.1 to 300+ PSI, and is controlled through signaling from the control unit 510. The valving mechanism 370 can comprise a one-way valve which may or may not include a pressure check component. The valving mechanism 370, in some variations, comprises complementary flaps which are biased in a closed position and which open in response to gas being expelled by the pressure source 505. In some cases, the pressure source 505 can be bi-directional in that it can apply both positive pressure or negative pressure (i.e., apply a vacuum/suction). The bi-directional aspect can be useful in cases in which certain fluid flow paths require priming and/or suction. Peristaltic and other types of pumps can be utilized to provide bi-directional fluid flow. In some cases, the pressure source 505 can comprise two or more pumps. In some cases, a first pump can provide positive pressure while a second pump can provide suction.

The control unit 510 comprises one or more processors (e.g., CPUs, GPUs, etc.) and memory storing instruction for execution by the one or more processors. The control unit 510 can include one or more programmable controllers (e.g., microcontrollers, etc.). The control unit 510 can execute operations or otherwise generate signals for execution by other components such as the pressure source 505 and the pressure sensor 520 as well as receive signals from the various components. For example, the control unit 510 can send signals to change pressure limits from a first value to a second value or to change infusion from a first profile (a first length of time at a first pressure) to a second profile (a second, different length of time at a second, different pressure).

In some variations, the control unit 510 can estimate and correlate pressures with volume infused, flow rate, and therapy settings relative to a type of the fluid and/or an infusion protocol. The control unit 510 can selectively send signals to the pump 510 to address various requirements including: maintaining a constant flow rate, varying flow rate, open or closed loop system control, biological signals that measure patient response during a therapy session, modifications of therapy parameters based on progression of patient along a therapy regime over the course of time, and other adjustable programs based on patient, clinician, pharmacy assessment of individual patient needs and the like. The control unit 510 can be programmed locally or remotely (e.g. on a user interface of a remote computing device in communication with the control unit 510 by way of the communications interface 525).

The pressure values generated by the pressure sensor 520 can be used by the control unit 510 to determine when all of the fluid in the medication container 120 has been expelled through the orifice 126. For example, the pressure remaining constant may indicate that all of the fluid has been expelled. In addition, the measured pressure can be used by the control unit 510 to determine if there is an occlusion condition within an infusion set (e.g., tubing 140 and/or needle set 150, etc.) coupled to the medication container 120. For example, spike or other pre-defined pattern in the measured pressure level may be indicative of an occlusion. The control unit 510 can, for example, use pattern matching or other signal processing techniques to correlate measured pressure profiles with empirically measured occlusion pressure profiles. Further, the control unit 510 can also determine, based on the measured pressure from the pressure sensor 520, that the medication container 120 has been removed from the medication container interface 340. For example, the pressure in the pressure chamber 160 may be vented when the medication container 120 has been removed which can be detected by the measured level of pressure by the pressure sensor 515.

The control unit 510 can be configured to allow for user-assisted or automatic priming of the medication container 120 to remove any bubbles and the like from the medication chamber 130 and from the fluid path to the patient. With the user-assisted approach, the user can activate the pressure source 505 (optionally through activation of the one of the buttons 320/330 and/or using a remote computing device such as a mobile phone, tablet, patient monitor, etc.) to expel fluid and/or gas causing the fluid to approach the terminus of the fluid path (i.e., the point at which fluid enters the patient, etc.). The terminus of the fluid path can, for example, be a needle(s) forming part of a needle set 150 as illustrated in FIGS. 1 and 2. In other cases, the terminus of the fluid path can be a needle or other injection device directly coupled to the infusion pump 110. The user can then deactivate the pressure source 505 once the user visually confirms that the fluid within the medication chamber 130 just prior to reaching the terminus of the fluid path. With the automatic approach, the control unit 510 can be configured to control the pressure source 505 to expel gas and/or fluid until such time as the fluid within the medication chamber 130 just prior to reaching the terminus of the fluid path. With such an arrangement, the control unit 510 can utilize a look up table corresponding to the type of medication container 120 and corresponding fluid attributes with likely priming requirements. In other variations, the control unit 510 can receive signals from a bubble sensor within or adjacent to the housing 310 which can optically detect whether there the medication chamber 130 is fully or adequately primed (i.e., bubbles have been expelled, etc.). In either approach, the control unit 510 can be cause the pressure source 505 to operate at a lower level than during a typical infusion to avoid or otherwise minimize the likelihood of fluid within the medication chamber 130 crystallizing when exiting the terminus of the fluid path.

The power source 515 can be a local power source (e.g., a rechargeable or non-rechargeable battery) or it can be a connector to an external power source (e.g., a hard-wired AC/DC adapter, etc.). In some cases, the amount of remaining charge of the power source 515 can be detected and conveyed to a user. Audio/visual alerts can be generated when the remaining charge is below a certain level and/or is insufficient to complete the current infusion.

The pressure sensor 520 can be positioned near or adjacent to the pressure source 505 and can measure pressure within the pressure chamber 160. In some variations, the system works in an open loop manner (as described in further detail below) in which case values generated by the pressure sensor 520 can be used to reference look up tables that establish flow rates as correlated with pressures given other attributes including, for example, biological parameters, temperature and accessories.

The communications interface 525 can be a wireless interface which allows the infusion pump to bi-directionally communicate over one or more networks and/or in a peer-to-peer fashion (e.g., Bluetooth, etc.) to external computing devices. In some variations, the communications interface 525 is a wired connection to a network, router, etc. The external computing devices which can exchange signals/data with the infusion pump 110 can take various forms including a mobile phone, tablet, remote computer, patient monitor, hospital data system and the like which can include an interface for monitoring, logging, and controlling infusions by the infusion pump 110 (e.g., change the operational parameters associated with the settings elements 330, etc.). The communications interface 525 in some variations can communicate with other external sensors or medical devices which are used in connection with the care of the patient receiving the infusion. Example sensors or medical devices include in-line flow sensors, in-line pressure sensors, physiological sensors measuring a patient vitals, and the like. In some cases, data received by way of the communications interface 525 from the external sensors/medical devices can be used by the control unit 510 to alter or otherwise change one or more operational parameters of the pneumatic pump which, in turn, alters how fluid within the medication container 120 is being infused to the patient. In some variations, the infusion pump 110 does not necessarily require the pressure sensor 520 and can obtain signals by way of the communications interface 525 from an external pressure sensor or flow rate sensor which can be positioned, for example, downstream from the medication container 120 along the tubing set 140.

The communications interface 525 can be used to transmit data characterizing the infusion such as fluid flow rate information or values to calculate the fluid flow rate information. Fluid flow rate information can take varying forms including one or more of time of infusion (as based on a clock of one of the components 505-530), fluid flow rate, fluid pressure profile (i.e., the pressure measurements during the course of a single infusion), mode of operation by the pressure source 505 (i.e., which setting element 330 was activated, etc.), and patient compliance statistics. The ability provided by the current subject matter to monitor patient therapy adherence each session and over time is an important adjunct to clinical optimization for each patient including prevention of medicine loss or misuse. Collection of statistical data generated by or in conjunction with the infusion pump 110 including therapy compliance and response for groups of patients can provide substantive enhancements to subsequent prescriptions as well as feedback to pharmacies about patient management and feedback to pharmaceutical companies about patient response to their respective drugs. In some cases, the communications interface 525 can receive signals external to the infusion pump 110 which would cause the control unit 510 to, in turn, cause the pressure source 505 to cease (thus ending an infusion).

The data store 530 can log information generated by the pressure source 505 and/or the pressure sensor 520 and the like in order to characterize infusion parameters. This logged information can be transmitted to a remote computing device by way of the communications interface 525 to, for example, track compliance with prescribed regimens and the like.

In some variations, the housing 310 can include an air port 395 which can be used to supply gas to a tubing set or other device (e.g., medication transfer device, wearable injectors, injector pen, etc.) coupled directly or indirectly to the air port 395.

The air port 395 can, in some variations, include a Luer lock to connect to such a tubing set or other device. Other connecting mechanisms can be provided depending on the downstream use of the air port. In some variations, the air port 395 is coupled to a tubing set extending therefrom. The gas supplied through the air port 395 can be by way of a channel coupled to the pump 510. This channel can be dedicated or selectively activated by way of valving within the housing. In other cases, the gas supplied through the air port can be generated by a second pump.

In some variations, a proximity sensor in or adjacent to the medication container interface 340 can be used which is configured to detect presence or absence of a medication container 120 within the medication container interface 340 and transmit corresponding signals to the control unit 510. The proximity sensor, in some implementations, can be an additional sensor 555 forming part of the component platform 380.

In some variations, a position sensor in or adjacent to the medication container interface 340 can be used which is configured to detect changes correlated to a volume of the medication chamber 130 (e.g., position of the stopper 124, etc.) and transmit corresponding signals to the control unit. The position sensor, in some implementations, can be an additional sensor 555 forming part of the component platform 380. The position sensor, in some variations, can visually determine and characterize movement of the plunger 124 (or other elements of the medication container 120 indicative of flow rate or remaining fluid, etc.) which can be used to calculate change in volume over time. The position sensor can be or include various sensors such as a linear displacement encoder, time-of-flight laser or ultrasound, or digital camera.

In some variations, the pressure sensor 520 can be omitted and a lookup table can be used (and optionally stored in the data store 530) which maps flow rate to viscosity of the fluid being administered.

The current subject matter is advantageous in that the infusion pump 110 can use medication containers 120 having varying sizes (and thus different barrel diameters). The force applied to a plunger 124 creates fluid pressure inside the medication container 120, which drives the fluid through orifice 126 to the tubing set 140/needle(s) 150. The resulting flow rate is directly influenced by this pressure. If the applied force remains constant but the barrel diameter changes, the fluid pressure varies inversely with the cross-sectional area of the barrel. Specifically, a larger barrel diameter reduces the pressure (and thus the flow rate), while a smaller diameter increases the pressure (and flow rate), due to the relationship P=F/A, where P is pressure, F is force, and A is the area of the syringe barrel.

By having the pressure source 505 generate constant air pressure as the driving mechanism (instead of a fixed mechanical force) different barrel sizes can be used as the air pressure applied to the fluid by way of the plunger 124 remains uniform regardless of the barrel diameter. This arrangement ensures that the fluid pressure, and consequently the flow rate, stays consistent even if the medication container barrel diameter varies, providing more reliable and predictable drug delivery.

The infusion pump 110 can have an electronic, closed-loop firmware control of the applied air pressure (by way of the pressure source 505) that can be adjusted via a user interface on a remote computing device or on setting elements 330. Air pressure applied to the plunger 124 (e.g., syringe seal, etc.) can be measured using the pressure sensor 520. The user-selected variable pressure setting (e.g., settings 1-5, etc.) allows a range of flow rates to be achieved without the need for flow control/restrictor devices along the tubing set 140. In some use cases, the applied pressure generated by the pressure source 505 can be fixed (not allowing the user to make adjustments). In some cases, the control unit 510 can cause the pressure generated by the pressure source 505 to be ramped, staged, or have different profiles depending on the infusion therapy requirements (e.g., ramped infusion rate or boluses). In particular, the setting elements 330 can provide user-selected variable pressure setting which allow a range of flow rates to be achieved without the need for flow control/restrictor devices. In some use cases, the applied pressure can be fixed (not allowing user to adjust). In some cases, the pressure can be ramped, staged, or have different profiles depending on the infusion therapy requirements (e.g., ramped infusion rate or boluses).

The use of near-constant applied air pressure (i.e., intermittent on/off of the pressure source 505 to maintain a set target pressure within a certain threshold/tolerance) with the use of feedback from the pressure sensor 520 allows for variable pressure control, volume estimation, flow rate control, syringe insertion/presence or removal detection, and end of infusion/occlusion detection, depending on the implementation and use application. The pressure source 505 duty cycle also helps to extend the device run time on a single charge of the internal battery (i.e., power source 515).

The control unit 310 can be used to control flow rate in either an open-loop or a closed-loop fashion, depending on the particular configuration and use application. Flow rate can be controlled by the control unit 310 modulating the applied pressure/force on the plunger 124 to achieve the desired flow rate. Flow rate can be measured or estimated by various techniques. In one variation, the flow rate can be determined based on the pressure applied and the viscosity of the medicine through a pre-recorded table (530). In another variation, flow rate can be determined using the ideal gas law equation (PV=nRT, PIV₁/T₁=P₂V₂/T₂) with pressure measurements and a known reference volume. In particular, a first pressure sensor 520 can measure pressure within the pressure chamber 160 and generate a first pressure value while a second pressure sensor 535 can measure pressure within a reference chamber 550 encapsulated within the housing 310 and generate a second pressure value. The control unit 310 can calculate an amount of fluid expelled from the medication container 120 based on a differential pressure measurement using the first pressure values and the second pressure value and leveraging the ideal gas law equation. In some cases, the temperature can be assumed to be the same. In other variations, a first temperature sensor 540 can measure temperature within the pressure chamber 160 and a second temperature sensor 545 can measure temperature within the reference chamber 550. Thus, the respective pressure and temperature values within the pressure chamber 160 and the reference chamber 550 can be used to calculate the volume of the pressure chamber 160 (which can then be correlated to amount of fluid expelled from the medication chamber 130).

In some variations, the housing 310 can encapsulate a pressure relief valve which is in communication with the pressure chamber 160. This pressure relief valve can, for example, be mechanical in that it releases pressure when a certain physical parameter condition (e.g., pressure value, etc.) is met. Alternatively, the pressure relieve valve can be electromechanical (e.g., a solenoid valve) which can be opened in response to a signal from the control unit 510. The pressure relief valve, in some variations, forms part of the component platform 380.

As noted above, while the current description is largely directed to the use of a plunger-less syringe, different types of medication containers 120 can be used. For example, the infusion pump 110 can be used in connection with an on-body injector which is adjustable to a contour of a body of a user. Such an on-body injector can have a dual chamber arrangement; namely a pressure chamber in combination with a medication chamber. With this arrangement, the air port 395 can be used to supply pressure to the pressure chamber. As another example, the medication container 120 can be an injector pen in which. The injector pen would also have a dual chamber in which the air port 395 is used to supply pressure to the corresponding. As yet another example, the medication container 120 can be a flat reservoir or bag in which pressure is applied directly or indirectly to a deformable/moveable surface of such reservoir or bag. In some variations, there is a dual chambers approach (pressure chamber in combination with medication chamber) with different form factors to elongate (pen) or flatten (onbody) as needed.

With the plunger-less syringe variation of the medication container 120 described above, the pressure generated by the pressure source 505 is directedly applied to the surface of the medication container 120 (i.e., the stopper 124). In other variations, pressure generated by the pressure source 505 is indirectly applied to the surface of the medication container 120 by way of one or more intermediate elements between the housing 310 and the medication container 120. For example, the pressure source 505 can inflate an expandable element (e.g., balloon, etc.) within the pressure chamber 160 which causes the fluid to be expelled out of the orifice 126 of the medication container 120. In the variation of a plunger-less syringe, the expandable element can contact the stopper 124.

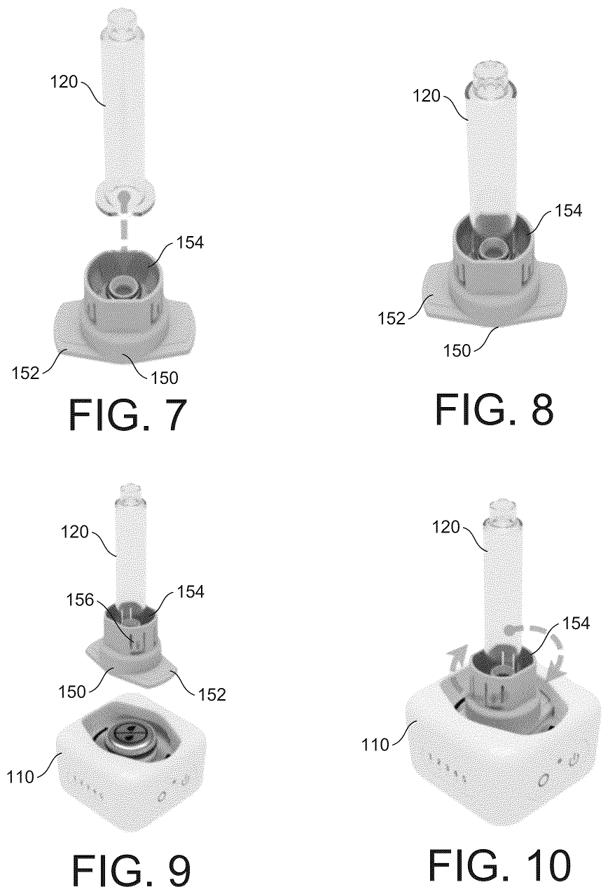

In some variations, the medication container 120 may have a shape and size that is not directly compatible with the medication container interface 310. In such cases and with reference to FIGS. 7-10, an adapter 150 can be utilized. The adapter can include flanges 152 extending therefrom to secure with the medication container interface. The adapter 150 can also include as a receiving cavity 154 into which the medication container is inserted. The housing of the adapter 150 can also include securing elements 156 (e.g., locking tabs, snaps, etc.) which can secure the medication container 120 after it is placed therein. The medication container 120 in FIGS. 7-10 omits the stopper 124 for ease of illustrated. FIG. 7 illustrates the medication container 120 and adapter 150 prior to coupling. FIG. 8 illustrates the medication container 120 after it is inserted into and secured by the adapter 150. FIG. 9 illustrates the combination of the medication container 120 and adapter 150 relative to the infusion pump. FIG. 10 illustrates the process of securing the medication container 120/adapter 150 combination into the infusion pump. Like the earlier examples, the medication container 120 and adapter 150 form a pressure chamber 160 relative to the mount 350 and seal 360.

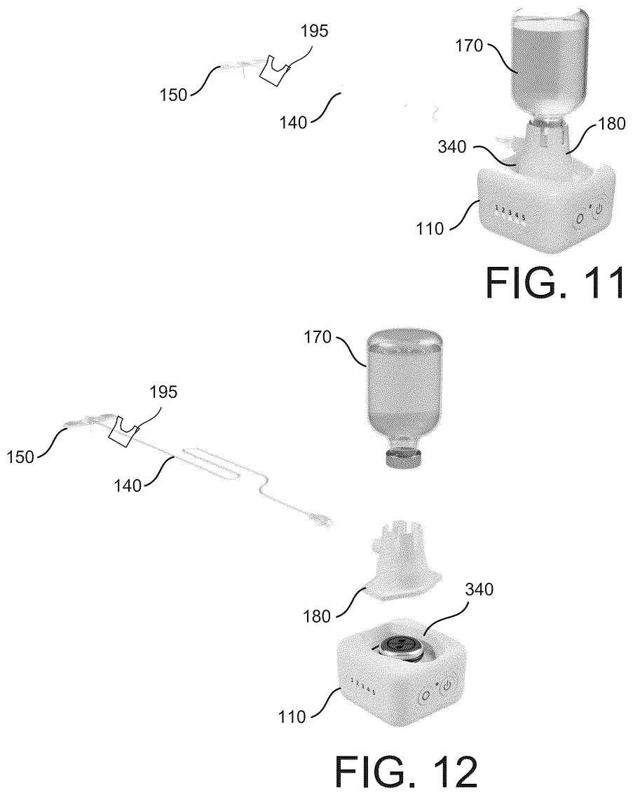

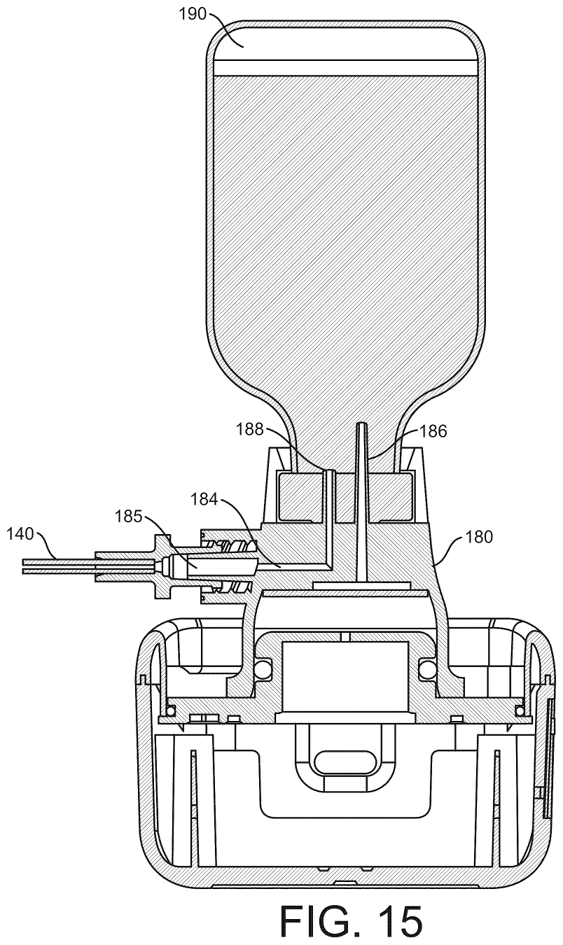

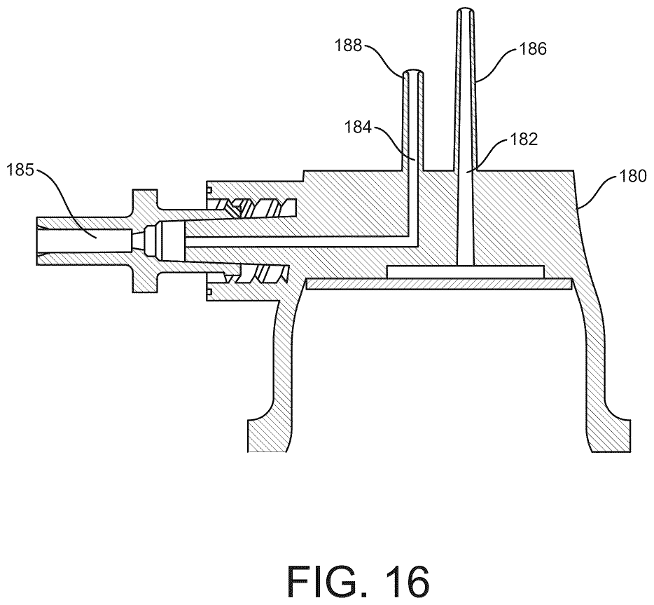

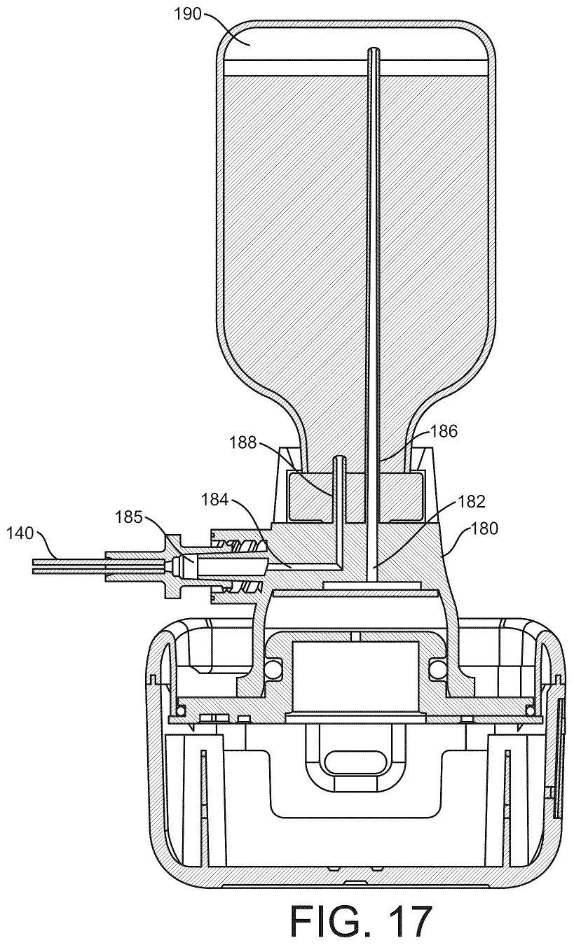

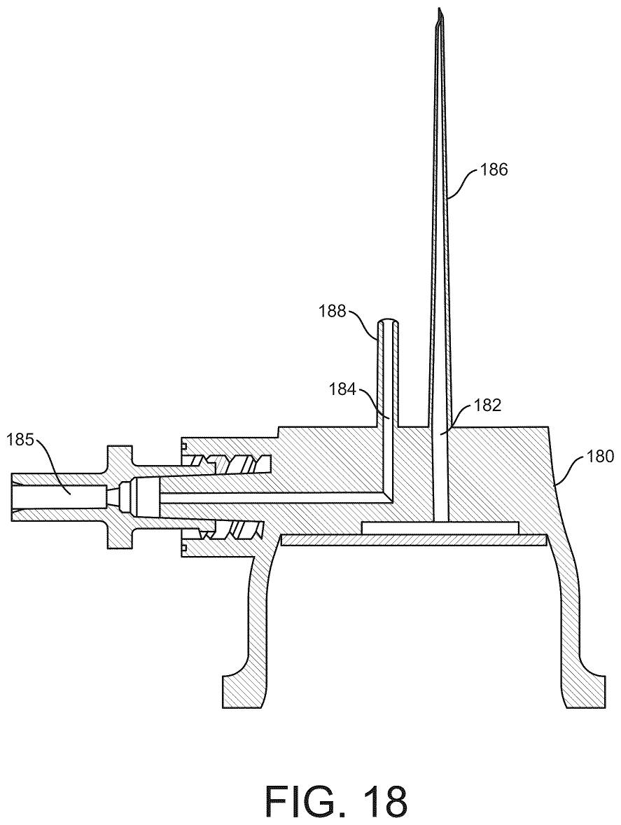

In some variations, and with reference to FIGS. 11-22, the medication container 120 can be a vial 170, with the opening of the vial secured directly to the medication container interface 340. Alternatively, a medication transfer device 180 can be attached to the opening of the vial 170 and then secured to the medication container interface 340. Different mechanisms can be used to secure the vial 170 to the medication container interface 340 including gripping members which are movable or deformable (and optionally biased inwards) and have a shape and size to extend around at least a portion of a flange of the vial 170. The medication transfer device 180 can, similar to the variations described above, include flanges or extending elements to allow it to secure to the medication container interface 340. The medication transfer device 180 can be used in connection with a wide variety of infusion pumps including pneumatic pumps (e.g., piezoelectric pumps, etc.), mechanical pumps (e.g., manually-activated mechanical pumps, etc.) and the like. The medication transfer device 180 can be disposable in that it is intended for one-time patient use. The medication transfer device 180 can include one or more spikes 186, 188, each shaped and sized to pierce the membrane or foil seal of the vial 170. These spikes 186, 188 allow the infusion pump 110, through the pressure source 505, to introduce gas into the vial 170 via an inlet channel 182. This pressurization can displace the fluid within the vial 170, causing it to exit through an outlet channel 184 and flow directly into the tubing 140 and/or needle set 150 (thus providing a direct-from-vial infusion path). The medication transfer device 180 can further include a Luer lock or other securing mechanism at the terminus 185 of the outlet channel 184 to provide a secure and fluid-tight connection with the tubing 140 or needle set 150. The outlet channel 184 can extend as close to as possible to the membrane (e.g., stopper, etc.) of the vial to help ensure that all fluid is expelled from the vial 170 (i.e., there is no pooling on the bottom of the vial when inverted). In some cases, the spike 188 can include perforations, other orifices, or grooves above the plane of the membrane to also help ensure that all fluid is expelled from the vial 170. In addition or in the alternative, in some variations the spike 186 can have perforations or other orifices along a portion of its body.

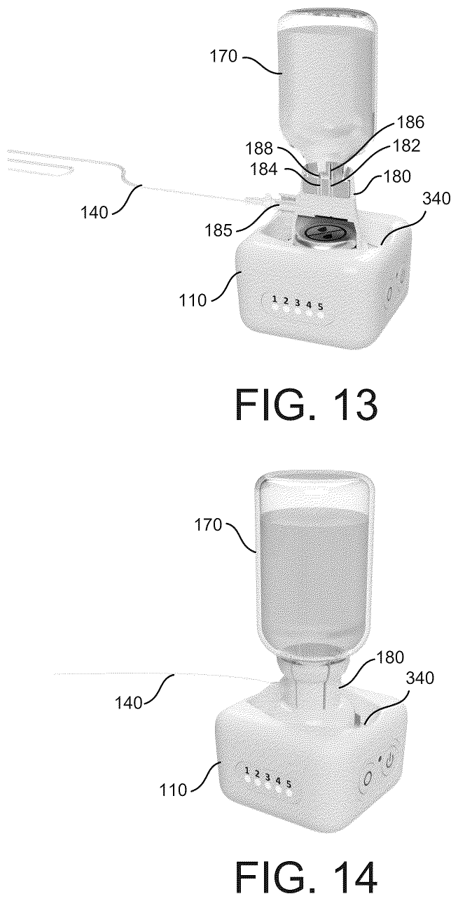

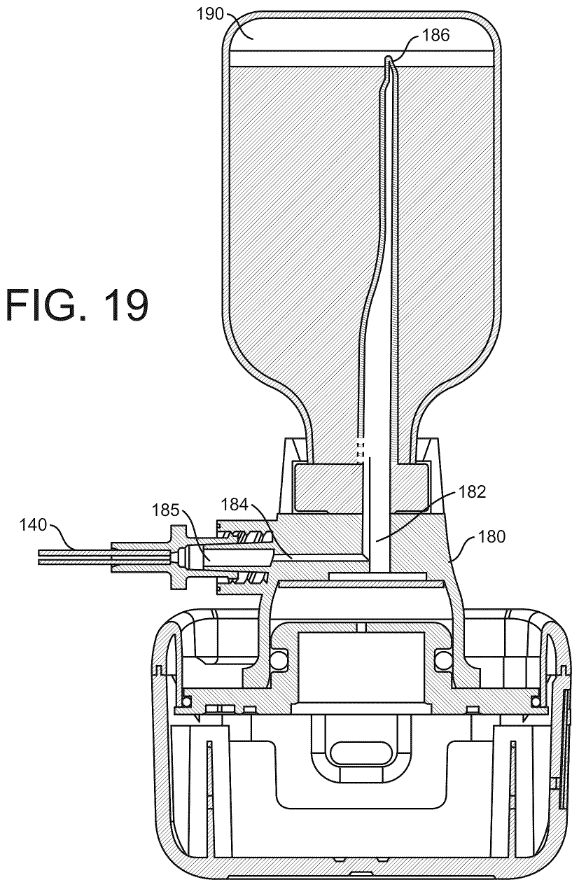

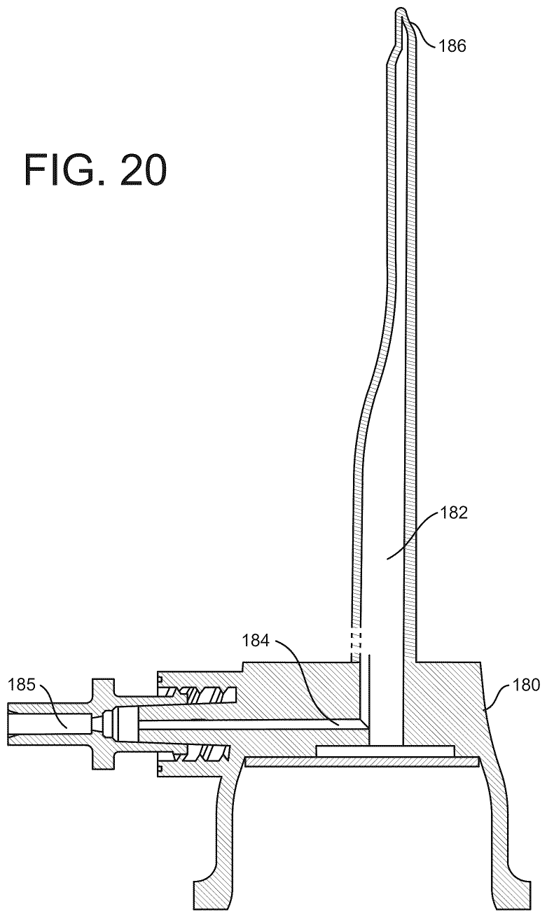

The medication transfer device 180 can, in some variations and as illustrated in FIGS. 13 and 15-18, include two spikes 186, 188 which respectively correspond to the inlet channel 182 and the outlet channel 186. These two spikes 186, 182 can have varying heights as illustrated. In some variations, the spike 182 corresponding to the inlet channel 182 can have a height that extends within the vial 170 (when inverted) to an air chamber 190 which helps expel fluid from the vial 170 through the outlet channel 186 at a desired and consistent rate and with minimal if any bubbles. In other variations, such as illustrated in FIGS. 19-20, a single spike 182 can be utilized that covers or otherwise encapsulates both of the inlet channel 182 and the outlet channel 184.

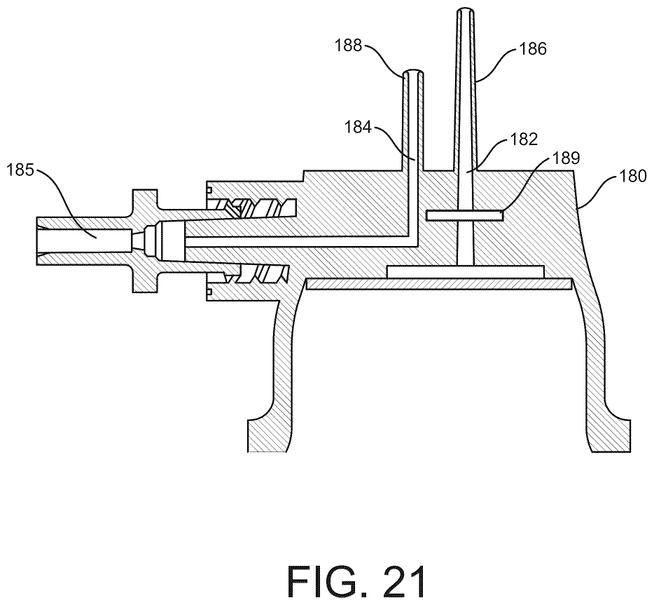

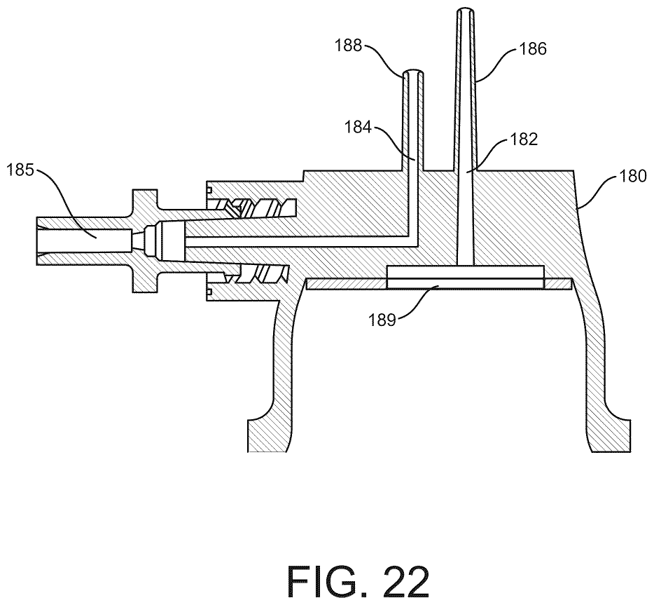

The medication transfer device 180 can, in some variations and as illustrated in FIG. 21 include a filter 189 disposed within the inlet channel 182 which can act to purify gas (e.g., air, etc.) passing therethrough. In other variations, as illustrated in FIG. 22, the filter 189 can be positioned on an outer face of the medication transfer device 180 (i.e., it can be intermediate the inlet channel 182 and the infusion pump, etc.).

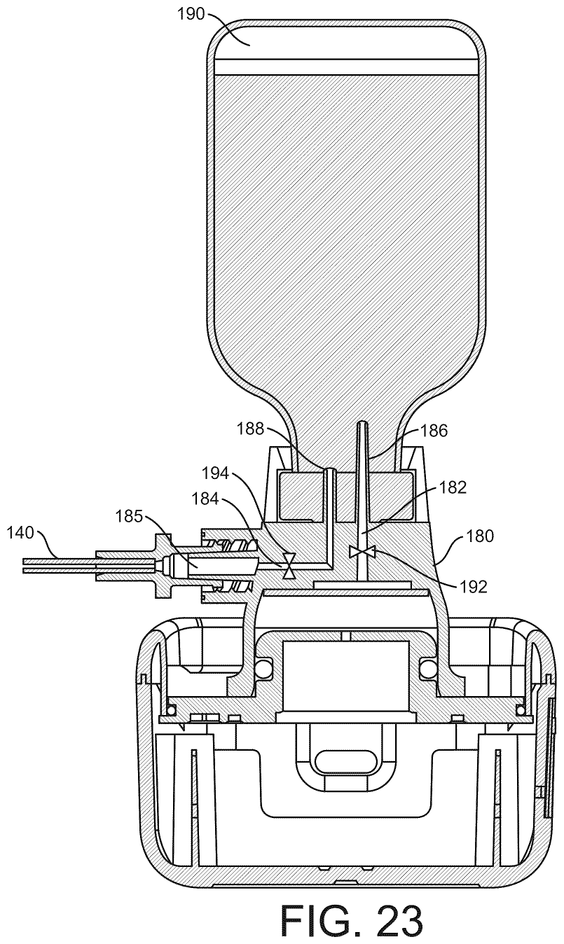

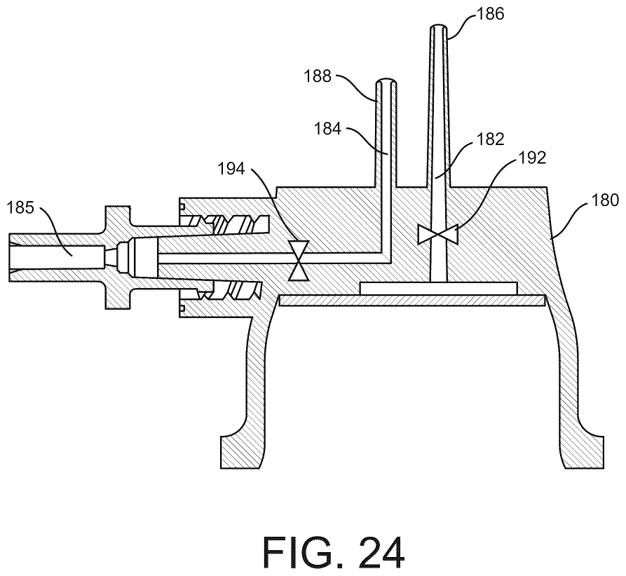

With reference to FIGS. 23-25, in some variations, the medication transfer device 180 can include one or more valves 192, 194 along the inlet channel 182 and/or the outlet channel 184. For example, a one way valve 192 within or upstream from the inlet channel 182 can prevent fluid within the vial 170 from exiting the vial through the inlet channel. In addition or in the alternative, a stop valve 194 within or downstream from the outlet channel 184 can selectively prevent fluid within the vial 170 and/or the medication transfer device 180 from flowing downstream from the outlet channel. In some implementations, one or more of the valves 192, 194 can be in communication with the control unit 510 so that the control unit 510 can send signals to selectively activate or close such valves 192, 194. For example, an electrical conduit can connect one of the valves 192, 194 to an electrical conduit on housing 310 (or equivalent for when the medication transfer device 180 is used with other pressure sources)

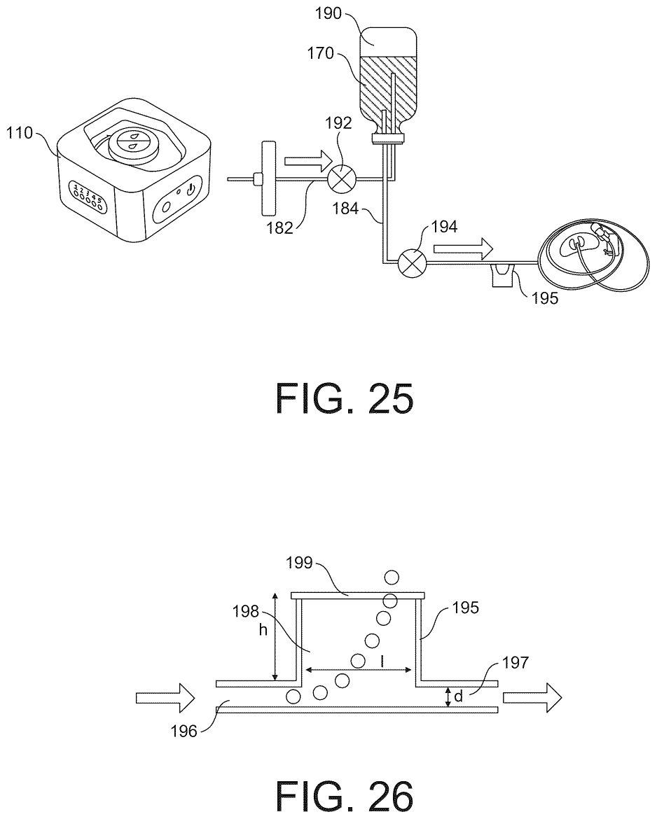

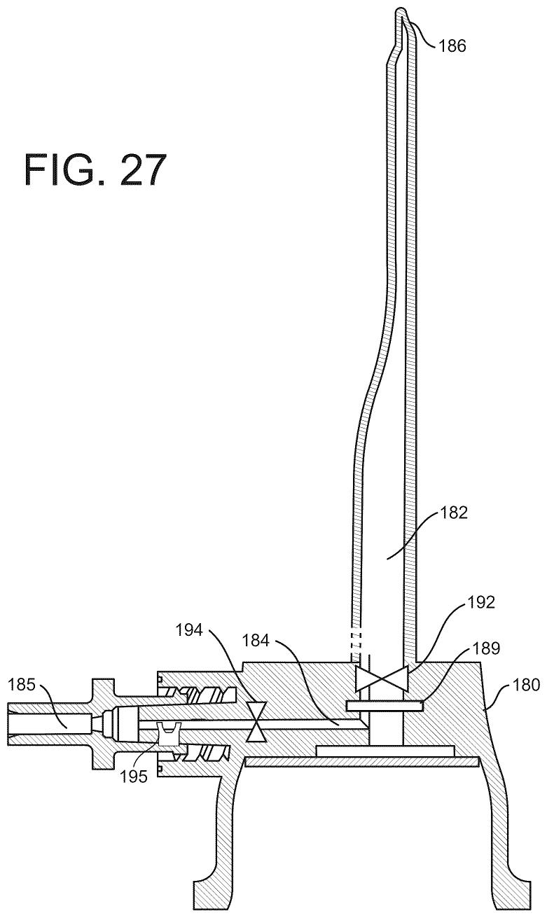

Also shown in FIG. 25 (and further detailed in FIG. 26) is a bubble eliminator 195 which can be downstream from the stop valve 194. In some cases, the bubble eliminator 195 is integrated into the medication transfer device 180, while in other cases, the bubble eliminator 195 is downstream from the medication transfer device 180 (see, for example, FIGS. 11 and 12). While the bubble eliminator 195 is illustrated in FIG. 25 as being used with both the one way valve 192 and the stop valve 194, it can also be incorporated into the flow stream independently of such valves and, in some cases, can be a standalone component with one or more connecting interfaces (e.g., Luer locks, etc.). The bubble eliminator 195 can include an inlet 196 which feeds fluid into a bubble chamber 198 and an outlet 197 by which fluid exits the bubble eliminator 195.

Intermediate the inlet 196 and the outlet 197 can be a bubble chamber 198 which has, one a least a portion of a surface, a permeable membrane 199. The height and length of the bubble chamber 198 can be configured so as to allow bubbles within the fluid entering the inlet 196 to rise and, in some cases, collect within the bubble chamber 198 (rather than exiting the outlet 197). The permeable membrane 199 allows for the bubbles to pass through while preventing fluid from passing through. The utilized permeable membrane 199 can be selected based on the desired pressure level of the fluid to be administered to a patient. Further details regarding features of the bubble eliminator can be found in U.S. patent application Ser. No. 63/820,593 filed on Jun. 9, 2025, the contents of which are hereby fully incorporated by reference.

In some variations, the pressure source 505 can include one or more pumps to effective give a bi-directional functionality of positive and negative pressure (i.e., vacuum). This can be accomplished through a dedicated two-way pump such as a peristaltic pump or through two single direction pumps working in concert and controlled by the control unit 510. The negative pressure setting can be particularly useful with regard to vials 170 in that it can help introduce air into the air chamber 190 by way of suction which then allows for fluid to be more effectively displaced from the vial 170 into the outlet channel 184 when the pressure source 505 is exerting positive pressure.

Various implementations and aspects of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor and memory, which may be special or general purpose, coupled to receive data and instructions from, and to transmit data and instructions to, a storage system, at least one input device, and at least one output device.

Although a few variations have been described in detail above, other modifications are possible. For example, the logic flow depicted in the accompanying figures and described herein do not require the particular order shown, or sequential order, to achieve desirable results. Other embodiments may be within the scope of the following claims.

1-14.

15. A medication transfer device comprising:
a first mechanical interface on a first end having a shape and size to securedly couple the medication transfer device to an infusion pump;
a second mechanical interface on a second end opposite the first end having a shape and size to secure a vial containing medication;
a single piercing element extending from the second end and defining both at least a portion of (i) an inlet channel in fluid communication with a pressure source generated by the infusion pump when the first interface is coupled to the infusion pump and (ii) an outlet channel in direct fluid communication with an infusion set delivering medication from the vial to a patient, the outlet channel providing a direct-from-vial infusion path in which medication is delivered from the vial directly to a patient without transfer to an intermediary container; wherein the medication transfer device is disposable and intended for one-time use.

16. The medication transfer device of claim 15 further comprising:
a one way valve within the inlet channel to prevent fluid within the vial from exiting the vial through the inlet channel.

17. The medication transfer device of claim 15 further comprising:
a stop valve within the outlet channel to selectively prevent fluid within the vial from flowing downstream from the outlet channel into the infusion set.

18. The medication transfer device of claim 17, wherein:
the stop valve is an electromechanical valve;
the medication transfer device comprises a first electrical conduit connected to the electromechanical valve and positioned to interface with a second electrical conduit on the infusion pump, when the medication transfer device is coupled to the medication transfer device;
the second electrical conduit is in communication with a control unit within the infusion pump; and
the stop valve is actuated by the control unit.

19. The medication transfer device of claim 17, wherein the stop valve is a duckbill valve.

20. The medication transfer device of 15 further comprising:
a bubble eliminator within the outlet channel having a bubble chamber and a hydrophobic air-permeable membrane through which bubbles within the bubble chamber pass.

21. The medication transfer device of claim 20 further comprising a housing forming the first mechanical interface, the second mechanical interface, and the single piercing element and encapsulating the bubble eliminator.

22. The medication transfer device of claim 15 further comprising: a filter on a first end of the inlet channel to purify gas passing therethrough.

23. The medication transfer device of claim 15 further comprising: a filter disposed within the inlet channel to purify gas passing therethrough.

24. The medication transfer device of claim 15 further comprising:
a one way valve within or upstream from the inlet channel to prevent fluid within the vial from exiting the vial through the inlet channel;
a stop valve within or downstream from the outlet channel to selectively prevent fluid within the vial from flowing downstream from the outlet channel;
a filter in the inlet channel to purify gas passing therethrough; and
a bubble eliminator in the outlet channel having a bubble chamber and a hydrophobic air-permeable membrane through which bubbles within the bubble chamber pass;
wherein the medication transfer device is disposable and intended for one-time use.

25. The medication transfer device of claim 15, wherein the single piercing elements comprises perforations along at least a portion of its body positioned to facilitate expulsion of all liquid in the vial.

26. The medication transfer device of claim 15, wherein the infusion pump comprises a pneumatic pump.

27. The medication transfer device of claim 15, wherein the infusion pump comprises a mechanical pump.

28. The medication transfer device of claim 27, wherein the mechanical pump is manually activated.

29-30. (canceled)

31. A medication transfer device comprising:
a vial transfer adapter having a unitary housing with (i) a first interface on a first end configured to mechanically couple the medication transfer device to an infusion pump and (ii) a second interface on a second end opposite of the first end configured to secure a vial containing medication; and
a single piercing element configured to pierce a stopper of the vial and defining at least a portion of both (iii) an inlet channel in fluid communication with a pressure source of the infusion pump and (iv) an outlet channel in direct fluid communication with an infusion set for delivering medication from the vial to a patient, the single piercing element comprising multiple side openings at different axial positions configured to facilitate evacuation of substantially all liquid from the vial when inverted; wherein the outlet channel provides a direct-from-vial infusion path in which medication is delivered from the vial directly to a patient without transfer to an intermediary container, and wherein the medication transfer device is disposable and intended for one-time use.

32. The medication transfer device of claim 31 further comprising a one-way valve within or upstream from the inlet channel to prevent liquid from the vial from exiting through the inlet channel.

33. The medication transfer device of claim 31 further comprising a stop valve within or downstream from the outlet channel to selectively prevent fluid flow from the vial into the infusion set.

34. The medication transfer device of claim 33, wherein the stop valve is electromechanical and the medication transfer device includes a first electrical conduit positioned to mate with a corresponding electrical conduit on the infusion pump to enable control of the stop valve by a control unit of the infusion pump.

35. The medication transfer device of claim 31 further comprising a bubble eliminator in the outlet channel having a bubble chamber and a hydrophobic air permeable membrane through which gas passes while liquid is kept within a fluid path in the outlet channel.

36. The medication transfer device of claim 31, wherein the outlet channel terminates in a tubing set interface comprising a Luer lock.

37-38. (canceled)

39. A medication infusion system comprising:
a housing defining a medication container adapter interface;
a pressure source within the housing;
a control unit in communication with the pressure source; and
a medication transfer device having a unitary housing shaped to mechanically interface with the medication container adapter interface and to secure a vial, the medication transfer device including a single piercing element configured to pierce a stopper of the vial and defining at least a portion of both (i) an inlet channel coupled to the pressure source and (ii) an outlet channel directly couplable to an infusion set;
wherein, when the pressure source is active, gas is expelled through the inlet channel into the vial to displace medication from the vial through the outlet channel for delivery to a patient via a direct-from-vial infusion path without transfer to an intermediary container.

40. The medication infusion system of claim 39, wherein the pressure source is bi directional to selectively apply positive pressure to expel fluid and negative pressure to draw gas into an air space of the vial prior to expelling fluid.

41. The medication infusion system of claim 39 further comprising a pressure sensor in communication with the control unit, the control unit being configured to detect at least one of an occlusion condition, end of fluid, or vial removal based on a measured pressure profile.

42. The medication infusion system of claim 39, wherein a bubble eliminator with a hydrophobic air permeable membrane is disposed within the outlet channel of the medication transfer device or downstream thereof.

43. The medication infusion system of claim 39, wherein the outlet channel includes a stop valve controlled by the control unit via an electrical conduit interface between the housing and the medication transfer device.

44. The medication infusion system of claim 39, wherein the inlet channel includes a one way valve that prevents liquid from the vial from exiting through the inlet channel.

45. The medication infusion system of claim 39, wherein the medication transfer device includes a filter within or at an upstream end of the inlet channel.

46. The medication infusion system of claim 39, wherein the medication transfer device is detachable and disposable and includes a Luer lock at a terminus of the outlet channel.

47. The medication transfer device of claim 15, wherein the single piercing element encapsulates both the inlet channel and the outlet channel within a single spike structure.

48. The medication transfer device of claim 15, wherein the single piercing element comprises at least three side openings disposed at different axial positions along a length of the single piercing element, wherein a distal-most side opening is positioned to remain immersed in medication when the vial is inverted and substantially depleted.

49. The medication transfer device of claim 15, wherein the second mechanical interface comprises a vial retention mechanism including at least one deflectable gripping member configured to engage a flange or neck portion of the vial to resist inadvertent separation.

50. The medication transfer device of claim 15, wherein the single piercing element comprises two spikes of varying heights, a first spike corresponding to the inlet channel and a second spike corresponding to the outlet channel, wherein the first spike extends to an air chamber within the vial when inverted.

51. The medication transfer device of claim 15, further comprising a filter disposed within the inlet channel to purify gas passing therethrough.

52. The medication transfer device of claim 15, further comprising a one-way valve within the inlet channel to prevent liquid from the vial from exiting through the inlet channel, and a stop valve within the outlet channel to selectively prevent liquid from flowing downstream.

53. The medication transfer device of claim 15, further comprising a bubble eliminator downstream from the outlet channel, the bubble eliminator comprising a bubble chamber and a hydrophobic air-permeable membrane through which bubbles pass while liquid is retained within a fluid path.

54. The medication transfer device of claim 15, further comprising a Luer lock at a terminus of the outlet channel to provide a fluid-tight connection with an infusion tubing set.

55. The medication transfer device of claim 15, further comprising a housing forming the first mechanical interface, the second mechanical interface, and the single piercing element, wherein the housing encapsulates a bubble eliminator within the outlet channel.

56. The medication transfer device of claim 15, wherein the infusion pump comprises a piezoelectric pneumatic pump, the piezoelectric pneumatic pump being in communication with a control unit that selectively sends signals to the piezoelectric pneumatic pump to control infusion parameters.

57. The medication transfer device of claim 15, wherein the infusion set is a subcutaneous infusion set configured for subcutaneous immunoglobulin therapy requiring controlled subcutaneous administration of medication at specific flow rates.

58. The medication transfer device of claim 17, wherein the stop valve is a duckbill valve positioned within the outlet channel to selectively prevent fluid within the vial from flowing downstream from the outlet channel into the infusion set.
